(19) United States
(12) Patent Application Publication
(10) Pub. No.: US 2007/0142376 A1
(43) Pub. Date: Jun. 21, 2007

(54) CONTROL OF INTRAOCULAR PRESSURE USING ALK5 MODULATION AGENTS

(75) Inventors: Debra L. FLEENOR, Crowley, TX (US); Iok-Hou PANG, Grand Prairie, TX (US); Allan R. SHEPARD, Fort Worth, TX (US); Mark R. HELLBERG, Arlington, TX (US); Abbot F. CLARK, Arlington, TX (US); Peter G. KLIMKO, Fort Worth, TX (US)

Correspondence Address: ALCON, IP LEGAL, TB4-8, 6201 SOUTH FREEWAY, FORT WORTH, TX, 76134, US

(73) Assignee: ALCON, INC., Hunenberg (CH)

(21) Appl. No.: 11/611,312

(22) Filed: Dec. 15, 2006

Related U.S. Application Data
(60) Provisional application No. 60/751,130, filed on Dec. 16, 2005.

(51) Int. Cl. A61K 31/5377; A61K 31/506; A61K 31/519; A61K 31/4745; A61K 31/4709; A61K 31/4439; A61K 31/444

(52) U.S. Cl. 514/235.2; 514/314; 514/337; 514/275; 514/341; 514/332; 514/249; 514/264.11; 514/300; 514/303

(57) ABSTRACT

An ophthalmic pharmaceutical composition useful in the treatment of glaucoma and control of intraocular pressure comprising an effective amount of a selective modulator of ALK5 receptor activity is disclosed. Also disclosed is a method of treating glaucoma and controlling intraocular pressure comprising applying a therapeutically effective amount of a pharmaceutical composition comprising a selective modulator of ALK5 receptor activity to an affected eye of a patient.

This application claims priority under 35 U.S.C. §119 to U.S. Provisional Patent Application No. 60/751,130 filed Dec. 16, 2006, the entire contents of which are incorporated herein by reference.

### TECHNICAL FIELD OF THE INVENTION

The present invention is related generally to treatments for glaucoma and more specifically to agents which selectively modulate the activity of the activin receptor-like kinase 5 (ALK5, or Type 1 TGF-β receptor) thereby lowering intraocular pressure such as that associated with glaucoma.

### BACKGROUND OF THE INVENTION

The eye disease glaucoma is characterized by a permanent loss of visual function due to irreversible damage to the optic nerve. The several morphologically or functionally distinct types of glaucoma are typically characterized by an undesirable elevation of intraocular pressure (IOP), which is considered to be causally related to the pathological course of the disease. Continuously elevated IOP has been associated with the progressive deterioration of the retina and the loss of visual function. In some cases, ocular hypertension, a condition in which IOP is elevated, can present without apparent loss of visual function. However, patients with ocular hypertension are considered to be at a high risk for eventually developing the visual loss associated with glaucoma. Therefore, lowering IOP can be an objective for the treatment of glaucoma patients and for patients with ocular hypertension in order to decrease the potential for, or severity of, glaucomatous retinopathy. Unfortunately, many individuals do not respond well when treated with existing glaucoma therapies.

Patients known as normotension or low-tension glaucoma patients have relatively low IOP, yet present with glaucomatous visual field loss. These patients may benefit from agents that lower and control IOP, because glaucoma that is detected early and treated promptly may have reduced or delayed loss of visual function. Conventional therapeutic agents that have proven to be effective for the reduction of IOP include both agents that decrease aqueous humor production and agents that increase the outflow facility. Such agents are in general administered by one of two routes; topically by direct application to the eye, or orally. However, many of these agents have associated side effects which may render them undesirable as ocular therapeutic agents.

The transforming growth factor-beta (TGF-β) family of cytokines includes multifunctional proteins that regulate production of a wide variety of gene products, and thus control a wide variety of cellular processes. For example, TGF-β family members are involved in inflammation, wound healing, extracellular matrix accumulation, bone formation, tissue development, cellular differentiation, and tumor progression, among others. [Barnard et al., Biochim Biophys Acta. 1990; Vol. 1032:79-87; Sporn et al., J Cell Biol., 1992; Vol. 119:1017-1021; Yingling et al., Nature Reviews, 2004; Vol. 3:1011-1022; Janssens et al., Endocr Rev., 2005; (epub ahead of print)]. Three mammalian isoforms have been identified to date: TGF-β1, TGF-β2, and TGF-β3, and these isoforms are structurally-similar, despite being encoded by different genes. [Massague J., Annu Rev Cell Biol., 1990; Vol. 6:597-641]

In aqueous humor (AH) collected from human eyes affected by primary open angle glaucoma (POAG), one of the most common forms of glaucoma in Western patients, various groups have reported significantly higher levels, compared to normal eyes, of the TGF-β2 isoform. [Tripathi et al., Exp Eye Res., 1994; Vol. 59:723-727; Inatani et al., Graefes Arch Clin Exp Ophthalmol., 2001; Vol. 239:109-113; Picht et al., Graefes Arch Clin Exp Ophthalmol., 2001; Vol. 239:199-207; Ochiai et al., Jpn J Ophthalmol., 2002; Vol. 46:249-253; Ozcan et al., Int Ophthalmol., 2004; Vol. 25:19-22]. The TGF-β2 isoform is also reported to increase extracellular matrix (ECM) production. [Kottler et al., Exp Eye Res., 2005; Vol. 80:121-134]. In POAG, a disproportionate accretion of ECM in the trabecular meshwork (TM) region of the eye is believed to impart greater resistance to AH outflow, resulting in increased IOP. [Rohen et al., Graefe's Arch Klin Exp Ophthalmol., 1972; Vol. 183:251-266; Lee et al., Trans Ophthalmol Soc UK., 1974; Vol. 94:430-449]. A direct link may therefore exist between elevated TGFPβ2 levels in AH and an elevated IOP.

### BRIEF SUMMARY OF THE INVENTION

The present invention in part relates to methods of treating glaucoma in human patients or other mammals. The present invention also relates to methods of lowering or controlling normal or elevated IOP in a human patient or other mammals.

Embodiments of the present invention control IOP and treat glaucoma by modulating the activity of the ALK5 receptor. In vitro, TGF-β2 acts on the ALK5 (Type 1 TGF-β receptor) resulting in increased production of extracellular matrix (ECM) proteins in the trabecular meshwork (TM). It is therefore postulated that the TGF-β2-induced increase in ECM production in the TM ultimately results in increased IOP in vivo. Downregulation of the effects of TGF-β2-mediated response(s) thus represents a potential means to lower and/or control IOP and treat glaucoma. For example, inhibition of ALK5 activity would be expected to lead to a reduction in TGF-β2-mediated ECM accumulation. Accordingly, if a compound that inhibits or otherwise selectively modulates the ALK5 receptor is introduced into such a system, the undesirable effects of TGF-β2 on IOP may be reduced or ameliorated.

Further, TGF-β isoforms 1, 2, and 3 belong to a family of cytokines which signal via transmembrane serine/threonine kinase receptors; other members of this superfamily include activins, inhibins, bone morphogenetic proteins, growth and differentiation factors and Mullerian inhibiting substance. The receptors for TGF-beta isoforms are grouped into two classes: Type I or activin-like kinase (ALK5 or ALK1) receptors and Type II receptors. TGF-β signaling is accomplished via Type II receptor phosphorylation of Type I receptors, e.g. ALK5, in the presence of TGF-β. Activated ALK5, in turn, phosphorylates the cytosolic proteins Smad2 and Smad3. Phosphorylated Smad2 and Smad3 proteins then form a complex with another Smad protein, Smad4. The resulting Smad protein complex subsequently translocates into the nucleus and drives gene transcription.

As used herein, the terms “selective ALK5 modulator” or “selective modulator” thus refer to an agent, other than inhibitory Smad proteins (e.g. Smad6 and Smad7), which inhibits either the activation/phosphorylation of ALK5 itself or which inhibits the ability of ALK5 to activate/phosphorylate its target Smad proteins. Such an agent preferentially inhibits ALK5 receptors over other ALK-type receptors, such as ALK3, which modulates signaling via bone morphogenic proteins. Such an agent also preferentially inhibits ALK5 receptors as compared to the Type II receptors or to other signaling kinases such as p38 MAPK. For example, GW-6604 has been reported to potently inhibit the phosphorylation of ALK5 (IC50˜0.14 μM), as compared to phosphorylation of TGF-β Type II receptors and p38 MAPK (IC₅₀'s of 10 μM and 9.5 μM, respectively). Brit J Pharmacol., 2005; Vol. 145:166-177.

Certain embodiments of the present invention comprise compositions or methods which include or use compounds capable of selective modulation of ALK5 receptor activity thereby modulating intraocular pressure in the eye. Interaction of cytokines, such as TGF-β2, or other compounds with the ALK5 receptor can result in changes in the production of extracellular matrix proteins in the trabecular meshwork, thereby modulating intraocular pressure. By modulating ALK5 receptor activity, subject compounds according to certain embodiments of the present invention are accordingly useful for lowering and/or controlling IOP associated with normal-tension glaucoma, ocular hypertension, and glaucoma, including primary open-angle glaucoma in humans and other warm-blooded animals. When used in such applications, the compounds may be formulated in pharmaceutical compositions suitable for topical delivery to the eye.

In yet another embodiment of the present invention, an in vitro method screens a selective modulator for ALK5 receptor activity. Such screening can assist with selecting new compounds for the treatment of glaucoma and control of IOP. The method comprises culturing trabecular meshwork cells in an appropriate growth medium. The cultured cells are split into replicate and/or experimental and/or control groups to which are added control solutions or experimental solutions comprising a selective modulator of ALK5 activity. Levels of extracellular matrix-related proteins, such as fibronectin, plasminogen activator inhibitor I (PAI-1), collagens, fibrillin, vitronectin, laminin, thrombospondin I, proteoglycans, or integrins, are then measured in each cell culture group. The extracellular matrix protein levels can then be compared between groups to determine the effect of experimental solutions comprising a selective modulator on ALK5 activity.

The foregoing brief summary broadly describes the features and technical advantages of certain embodiments of the present invention. Additional features and technical advantages will be described in the detailed description of the invention that follows. Novel features which are believed to be characteristic of the invention will be better understood from the detailed description of the invention when considered in connection with any accompanying figures. However, figures provided herein are intended to help illustrate the invention or assist with developing an understanding of the invention, and are not intended to be definitions of the invention's scope.

### BRIEF DESCRIPTION OF THE DRAWINGS

A more complete understanding of the present invention and the advantages thereof may be acquired by referring to the following description, taken in conjunction with the figures of the accompanying drawing in which like reference numbers indicate like features and wherein:

FIG. 1 is a graph of results showing the effects of infused TGF-β2 on the IOP of a perfused human anterior segment model compared to control;

FIG. 2 is a graph of results showing the effect of an ALK5 inhibitor on fibronectin levels in a TGF-β2-treated perfused human anterior segment model compared to control;

FIG. 3 presents graphs showing measured levels of fibronectin and PAI-1 in in vitro TM cell cultures to which various concentrations of an ALK5 inhibitor have been added; and

FIG. 4 presents graphs showing measured levels of pro-collagen type I C-peptide (PIP) in in vitro TM cell cultures.

### DETAILED DESCRIPTION OF THE INVENTION

Certain embodiments of the present invention comprise compounds, compositions, or methods which include or use compounds capable of selective modulation of ALK5 receptor activity, thereby modulating intraocular pressure in the eye. Specific representative compounds that have been found to possess ALK5 modulating activity are listed below. In preferred embodiments, compounds for practicing the method of the present invention comprise compounds 1 and 2, shown below. In yet other embodiments, one or more of the following compounds may be used:

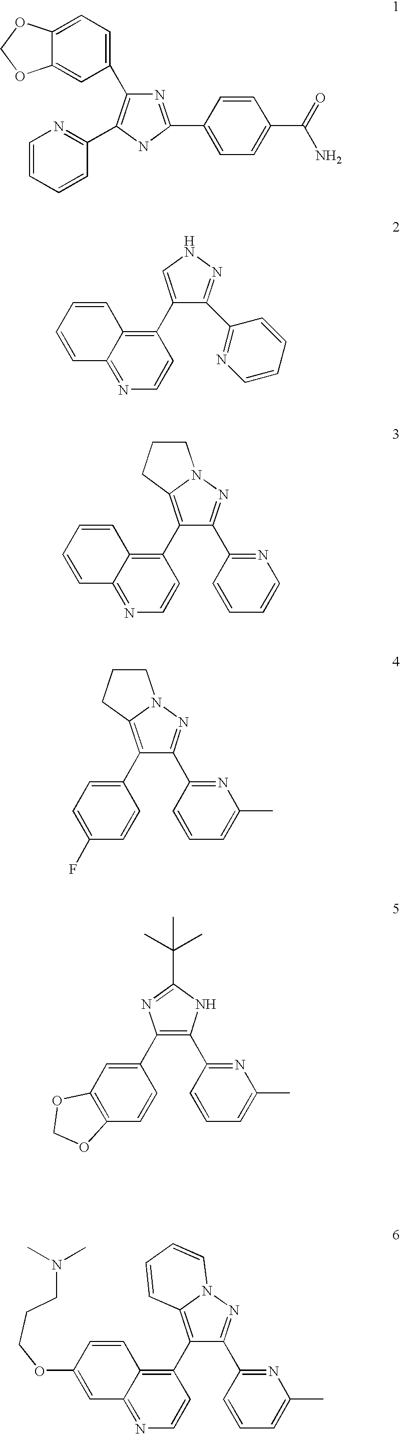

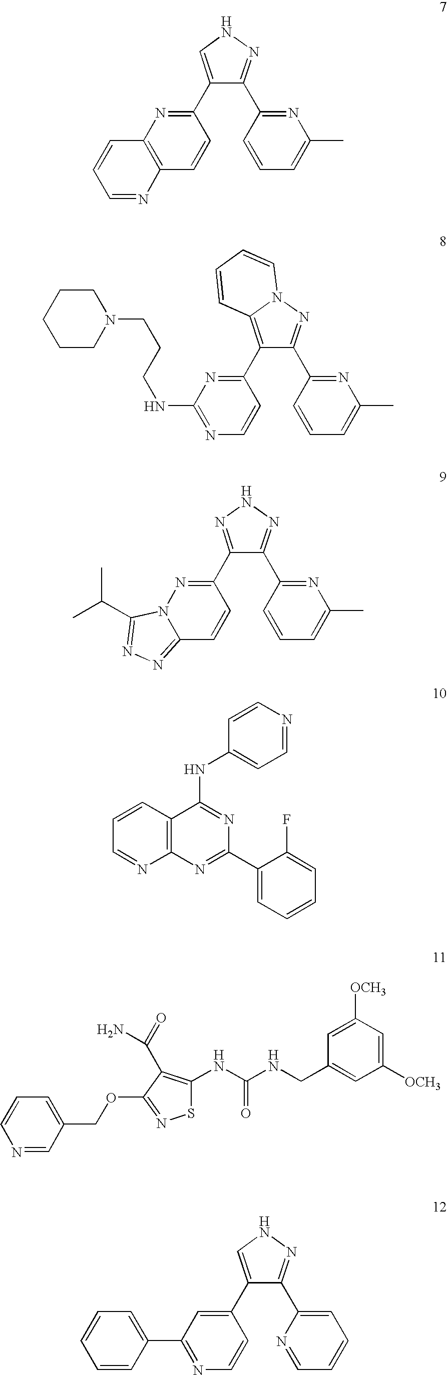

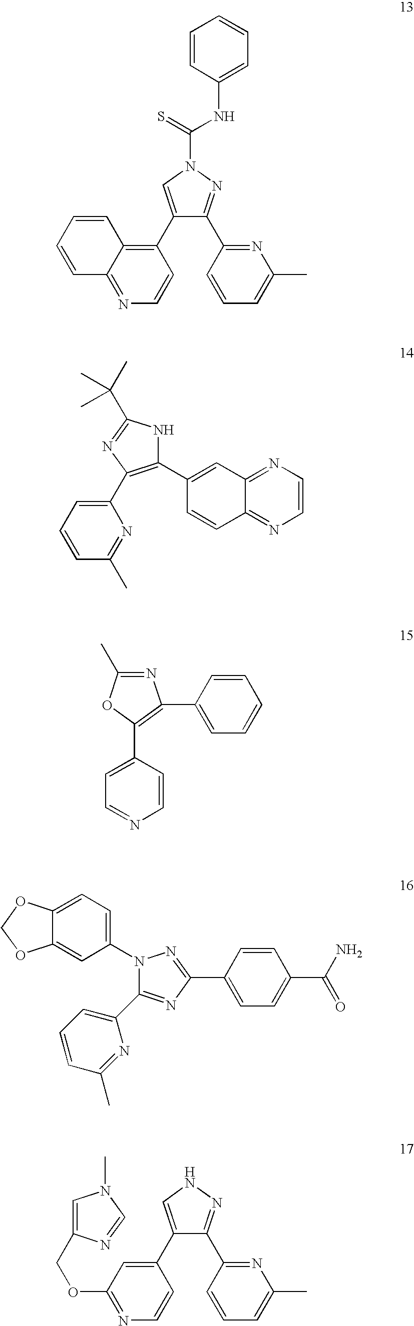

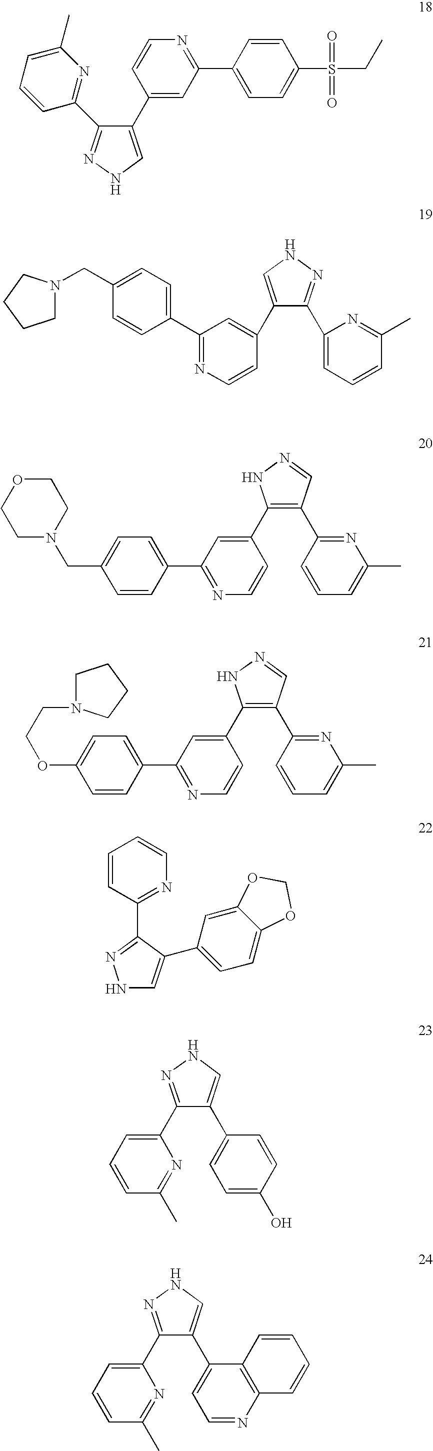

Certain compounds shown above may be referenced by a manufacturer designation. These include compound 1 (SB-431542), compound 2 (LY-364947), compound 3 (LY-550410), compound 4 (LY-580276), compound 5 (SB-504124), compound 12 (GW-6604), compound 13 (A-83-01), compound 14 (SB-525334), and compound 15 (SC-68376). In addition to the above compounds, or in other embodiments, one or more of the following compounds listed in Groups I and II below may be used:

Group I:

4-(3-(6-methyl pyridin-2-yl)-1H-pyrazol-4-yl)-7-ethoxy quinoline; 4-(3-pyridin-2-yl-1H-pyrazol-4-yl)-7-ethoxyquinoline; 7-fluoro-4-[3-(6-methyl-pyridin-2-yl)-1H-pyrazol-4-yl]-quinoline; 4-[3-(6-bromopyridin-2-yl)-1H-pyrazol-4-yl]-quinoline; 4-[3-(6-[n-butylamino)pyridin-2-yl]-1H-pyrazol-4-yl]-quinoline; 4-[3-(6-methylpyridin-2-yl)-1H-pyrazol-4-yl]-quinoline; 6-chloro-4-[3-(6-methylpyridin-2-yl)-1H-pyrazol-4-yl]-quinoline; 6-trifluoromethyl-4-[3-(6-methylpyridin-2-yl)-1H-pyrazol-4-yl]-quinoline; 7-methyl-4-[3-(6-methylpyridin-2-yl)-1H-pyrazol-4-yl]-quinoline; 6-methoxy-4-[3-1H-pyrazol-4-yl]-quinoline; 6-trifluoromethoxy-4-[3-(6-methylpyridin-2-yl)-1H-pyrazol-4-yl]-quinoline; 4-[3-(3-chlorophenyl)-1H-pyrazol-4-yl]-quinoline; 6-butoxy-4-(3-pyridin-2-yl-1H-pyrazol-4-yl)-quinoline; 6-sec-butyl-4-(3-pyridin-2-yl-1H-pyrazol-4-yl)-quinoline; 5-methyl-3-(6-methylpyridin-2-yl)-4-(-4-fluorophenyl)-1H-pyrazole; 4-(4-methoxyphenyl)-5-methyl-3-(6-methylpyridin-2-yl)-1H-pyrazole; 4-[5-methyl-3-(6-methylpyridin-2-yl)-1H-pyrazol-4-yl]-quinoline; 4-[3-(6-propylpyridin-2-yl)-1H-pyrazol-4-yl]-quinoline; 3 -cyclopropyl-5-pyridin-2-yl-4-quinolin-4-yl-pyrazole; 3-(3-trifluoromethylphenyl)-4-quinolin-4-yl-pyrazole; 1-benzyl-3-(2-pyridyl)-4-(4-quinolyl)pyrazole; 1-(4-phenylbutyl)-3-(2-pyridyl)-4-(4-quinolyl)pyrazole; 2-(3-(2-pyridyl)-4-(4-quinolyl)pyrazolyl)ethan-1 -ol; 2-(3 -(2-pyridyl)-4-(4-quinolyl)pyrazolyl)ethyl methylsulfonate; 4-[2-(3-(2-pyridyl)-3-(4-quinolyl)-pyrazolyl)ethyl]morpholine; phenyl[2-(3-(2-pyridyl)-4-(4-quinolyl)-pyrazolyl)ethyl]amine; 4-(4-pyridin-2-yl-1H-pyrazol-3-yl)-quinoline; and 4-(3-pyridin-2-yl-1H-pyrazol-4-yl)-quinoline.
   Group II:
5-[5-(6-methylpyridin-2-yl)-1H-[1,2,3]triazol-4-yl]-benzo[1,2,5]thiadiazole; 5-[2-ethyl-5-(6-methylpyridin-2-yl)-2H-[1,2,3]triazol-4-yl]-benzo[1,2,5]thiadiazole; 6-[5-(6-methylpyridin-2-yl)-1H-[1,2,3]triazol-4-yl]-[1,2,4]triazolo[1,5-a]pyridine; 2-[5-(2,3-dihydrobenzofuran-5-yl)-3H-[1,2,3]triazol-4-yl]-6-methylpyridine; 2-[5-(2,3-dihydrobenzo[1,4]dioxin-6-yl)-2H-[1,2,3]triazol-4-yl]-6-methylpyridine; 1 -methyl-6-[5-(6-methylpyridin-2-yl)-2H-[1,2,3]triazol-4-yl]-1H-benzimidazole; 6-(2-ethyl-5-(6-methylpyridin-2-yl)-2H-[1,2,3]triazol-4-yl)-[1,2,4]triazolo[1,5-a]pyridine; 6-(2-methyl-5-(6-methylpyridin-2-yl)-2H-[1,2,3]triazol-4-yl)-[1,2,4]triazolo[1,5-a]pyridine; 2-[5-(4-Methoxyphenyl)-2H-[1,2,3]triazol-4-yl]-6-methylpyridine; 2-[5-(3-fluoro-4-methoxyphenyl)-2H-[1,2,3]triazol-4-yl]-6-methylpyridine; and 2-[5-(3-chloro-4-methoxyphenyl)-2H-[1,2,3]triazol-4-yl]-6-methylpyridine.

From the collection of compounds described above, the following can be obtained from commercial sources: 1, commercially available from Sigma, P.O. Box 14508, St. Louis, Mo., 63178-9916; 2, commercially available from Matrix Scientific, P.O. Box 25067, Columbia, S.C., 29224-5067; and 15, commercially available from G. Scientific, Inc., 6450 Lusk Blvd. Suite E102, San Diego, Calif., 92121.

The other compounds can be synthesized as described in source references as follows [format: compound number(s), synthesis reference]:
3 and 4, Sawyer et al., Bioorganic and Medicinal Chemistry Letters, 2004; Vol. 14:3581-3584;

5 and 14, WO 2001/062756A1;

6, WO 2004/026871;

7, Gellibert et al., Journal of Medicinal Chemistry, 2004; Vol. 47:4494-4506;

8, WO 2004/021989;

9, WO 2004/026307;

10, WO 2000/012497;

11, WO 2004/147574;

16, Kim et al., Bioorganic and Medicinal Chemistry Letters, 2004; Vol. 12: 2013-2020;

12, WO 2002/066462;

13, Tojo et al., Cancer Science, 2005; Vol. 96:791-800;

17-21, WO 2004/016606;

22, U.S. Patent Application Publication No. 2004/116474;

23 and 24, Sawyer et al., Journal of Medicinal Chemistry, 2003; Vol. 46:3953-3956;

Group I compounds, WO 2004/026302; and

Group II compounds, U.S. Patent Application Pub. No. US 2004/152738.

The representative compounds above are in no way intended to limit the scope of the invention. The scope of the invention comprises any agents which may be identified as having the ability to selectively regulate, inhibit, or modulate the activity of the activin receptor-like kinase 5 (ALK5; or Type I TGF-β receptor).

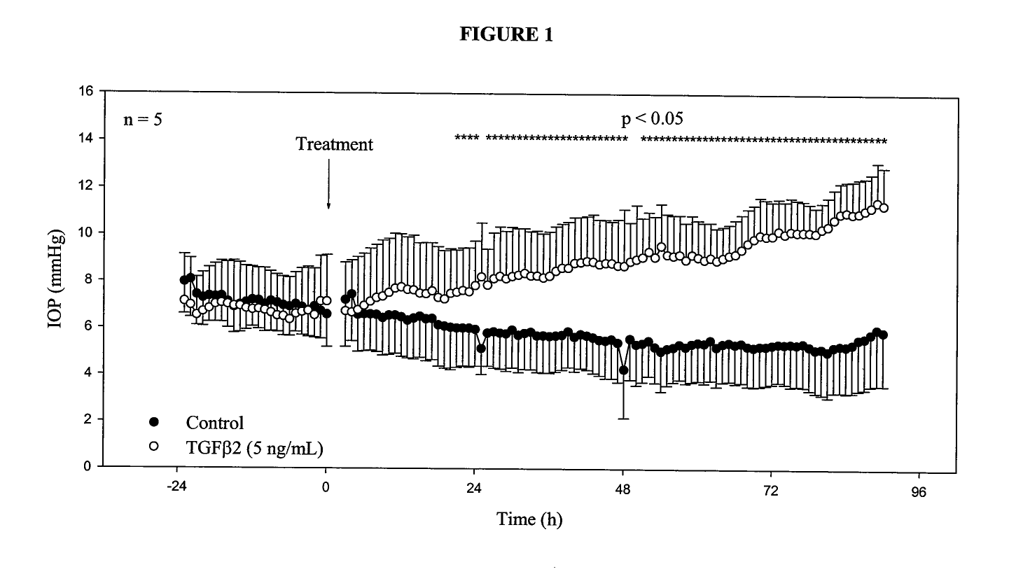

FIG. 1 is a graph showing the effect of infused TGF-β2 on a perfused human anterior segment model. All donor eyes used in this model were used according to the provisions of the Declaration of Helsinki for research involving human tissue, and were used within 24 hours post-mortem. No donors were known to have a history of glaucoma or other ocular disorder.

Human ocular perfusion organ culture was performed as described in available literature. [Tschumper et al., Curr Eye Res., 1990; Vol. 9:363-369; Clark et al., Invest Ophthalmol Vis Sci., 1995; Vol. 36:478-489; Pang et al., J Glaucoma, 2000; Vol. 9:468-479; Pang et al., Invest Ophthalmol Vis Sci., 2003; Vol. 44:3502-3510]. Briefly, anterior segments were dissected and mounted into custom Plexiglas culture chambers, then perfused with serum-free Dulbecco's modified Eagle's medium. IOP was monitored every 5 seconds and averaged each hour. Perfused tissue was allowed to equilibrate at 37° C. and 5% CO₂ until a stable baseline IOP was achieved, typically 2-4 days; tissues with unstable IOP were discarded. Stable tissues were then further perfused with media containing the test compound(s) as indicated and changes in IOP were recorded. Eluate samples were collected daily for ELISA analysis of fibronectin and PAI-1 content. Tissues were fixed and evaluated for viability/morphology by light and electron microscopy at termination of each study. Data from unacceptable tissues were excluded from results. Criteria for “unacceptable” tissues included findings such as excess debris in the TM region, denudation of TM beams, loss of TM and/or Schlemm's canal cells, and breaks or collapse of Schlemm's canal.

The results shown in FIG. 1 indicate that a perfused human anterior segment model infused with TGF-β2 at 5 ng/mL resulted in elevated IOP within 24 hours when compared to a control. IOP of the model receiving the TGF-β2 infusion was almost double that of the control after 72 hours.

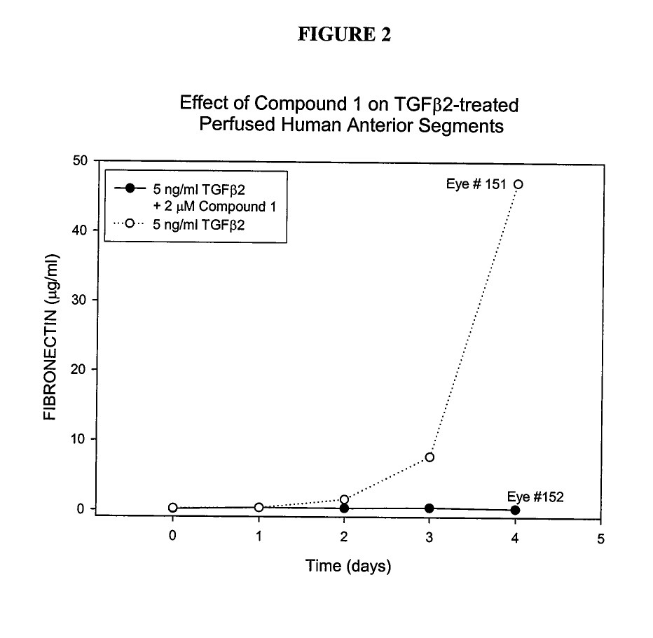

As postulated above, the introduction of compounds with selective ALK5 modulation activity reduces or ameliorates the undesirable effects of TGF-β2-induced ECM production. In FIG. 2, experimental results are presented showing decreased fibronectin levels in perfusates from human anterior segments treated with TGF-β2 and compound 1, shown below, compared to a control model perfused with only TGF-β2. Compound 1 completely antagonized TGF-β2-mediated increase in perfusate fibronectin content.

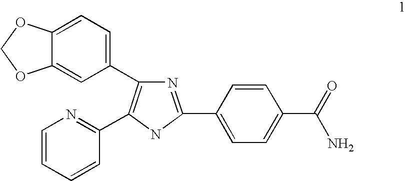

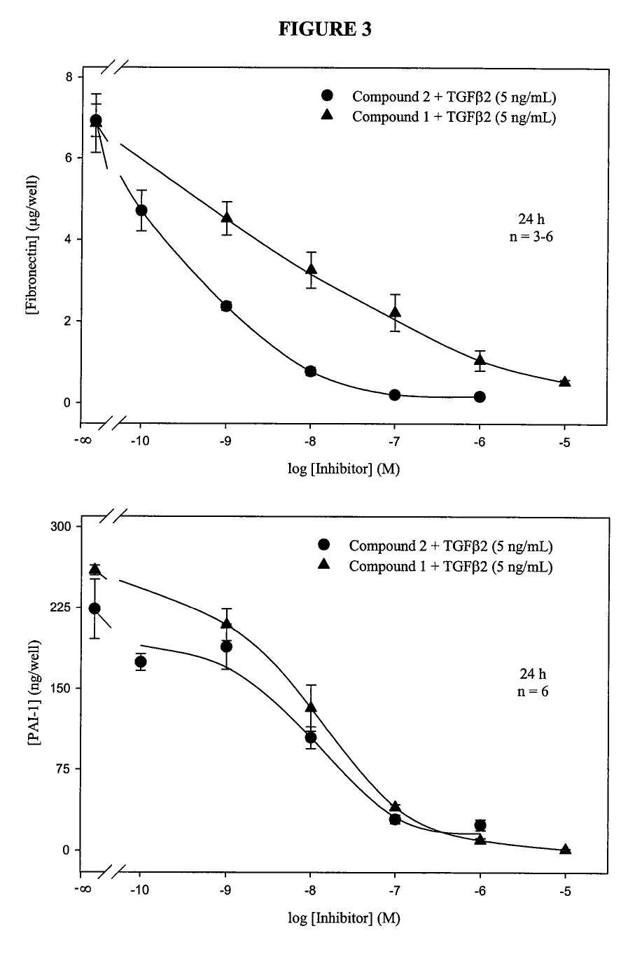

FIG. 3 shows graphs summarizing results of a study using cultured human TM cells. Generation and characterization of the GTM-3 transformed cell line has been previously described (Pang et al., Curr Eye Res., 1994; Vol. 13:51-63). Briefly, maintenance growth medium consisted of Dulbecco's modified Eagle's medium with Glutamax I (Gibco/BRL, Grand Island, N.Y.) supplemented with 10% fetal bovine serum (Hyclone, Logan, Utah) and 50 μg/mL gentamicin (Gibco/BRL). For assay, cultures were trypsinized and seeded into 24-well plates (Coming Costar, Acton, Mass.) and allowed to grow until monolayers reached approximately 90% confluence. Culture medium was then replaced with 0.25 mL serum- and antibiotic-free medium containing the appropriate test compound(s). Cells were incubated 24 h, at 5% CO₂ and 37° C. Aliquots of culture supernatants were then assayed for fibronectin and/or PAI-1 content by ELISA.

The study results shown in FIG. 3 reveal a dose-dependent inhibition of TGF-β2-mediated increase in fibronectin and PAI-1 content in supernatants from human TM cell cultures by ALK5-modulating compounds 1 and 2.

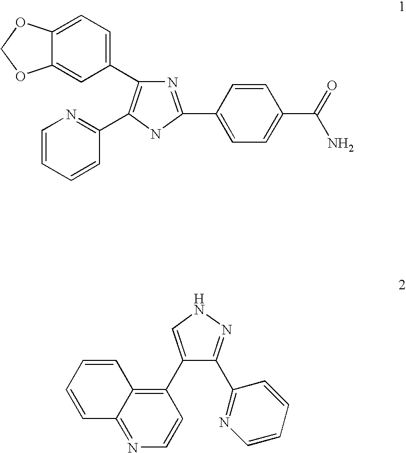

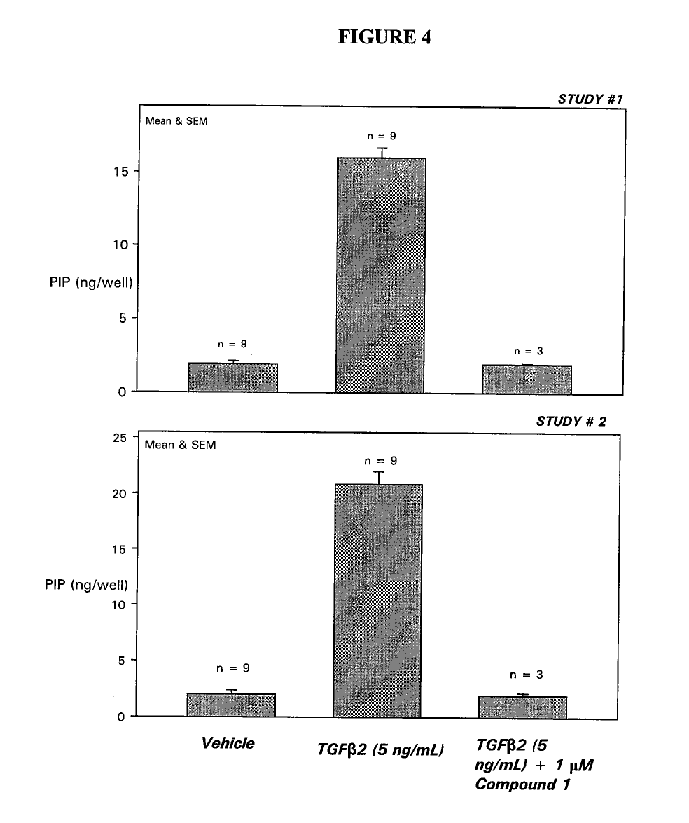

FIG. 4 shows graphs summarizing measured pro-collagen type 1 C-peptide (PIP) levels in human TM cell cultures. For this experiment, cultured transformed GTM-3 cells (Pang et al., Curr Eye Res., 1994; Vol. 13:51-63) were grown in a growth medium consisting of Dulbecco's modified Eagle's medium with Glutamax I (Gibco/Invitrogen, Grand Island, N.Y.) supplemented with 10% fetal bovine serum (Hyclone, Logan, Utah) and 50 μg/mL gentamicin (Gibco/Invitrogen). For assay, cultures were enzymatically-dissociated (TrypLE Express; Gibco/Invitrogen) then seeded into 24-well plates (Corning Costar, Acton, Mass.) and allowed to grow until monolayers reached approximately 90-95% confluence. Culture medium was then replaced with 0.25 mL serum- and antibiotic-free medium containing the appropriate test compound(s). Cells were incubated 24 h, at 5% CO₂ and 37° C. Aliquots of culture supernatants were then assayed using an ELISA kit for procollagen Type I C-peptide (TaKaRa Bio, Shiga, Japan).

Collagens are synthesized as pro-collagens, most of which contain additional peptide sequences called “propeptides”. Propeptides are located at both the N—and C—terminal ends of the molecules. These propeptides serve to facilitate formation of the mature collagen's triple helical structure from pro-collagens within the endoplasmic reticulum. The propeptide portions are then cleaved from the triple helix collagen molecules upon secretion—thus concentration of free propeptide, such as PIP, can be used to correlate changes in the amount of collagen being synthesized by cells. The results from both study replicates show that PIP levels are greatly elevated in TGF-β2-treated cultures compared to vehicle. However, when cultures are treated with both TGF-β2 and the ALK5 modulator Compound 1, this TGF-β2-dependent PIP elevation is eliminated. Thus, the study results shown in FIG. 4 demonstrate inhibition of TGF-β2-mediated increases in PIP levels by ALK5-modulating Compound 1. Given that PIP levels are directly linked to collagen production, an ALK5-modulator such as Compound 1 appears to decrease collagen production, and accordingly should inhibit overall ECM protein production in the TM.

TABLE 1, shown below, summarizes the results of a study measuring the effect of TGF-β2 on ECM-related protein levels (fibronectin, PAI-1) in cultured TM cells of various strains. TGF-β2 was present in the cultures at a concentration of 5 ng/TnL, and protein levels (mean±s.e.m.) were measured after 24 hours. The table results indicate that TGF-β2 increases the production of fibronectin and PAI-1 in a variety of human TM cell cultures.

TABLE 1
Effect of TGF-β2 on HTM Cell Secretion of Fibronectin and PAI-1

| | Fibronectin (μg/well) | | | | PAI-1 (ng/well) | | | |
|---|---|---|---|---|---|---|---|---|
| | | | | Fold | | | | Fold |
| Cell Strain | n | Control | TGF-β2* | Increase | n | Control | TGFβ2* | Increase |
| GTM-3 | 219 | 3.1 ± 0.3 | 16.7 ± 1 | 5.4 | 71 | 13.7 ± 0.8 | 266 ± 8 | 19.4 |
| NTM25-91 | 4 | 3.1 ± 0.4 | 19.3 ± 1.5 | 6.2 | | | | |
| NTM35 | 7 | 3.2 ± 1.8 | 13 ± 2.7 | 4.1 | | | | |
| NTM553-02 | 14 | 1.5 ± 0.2 | 10.1 ± 1.1 | 6.7 | 10 | 128.8 ± 1.7 | 315.9 ± 9.5 | 2.5 |
| NTM974-03 | 9 | 3.9 ± 1.1 | 8.9 ± 1.5 | 2.3 | 6 | 107 ± 4.1 | 297.7 ± 23.1 | 2.8 |
| NTM875-03 | 10 | 0.5 ± 0.3 | 9.6 ± 3.8 | 19.2 | 10 | 109.1 ± 7.9 | 282.6 ± 11.6 | 2.6 |
| GTM29-01 | 10 | 1 ± 0.2 | 9.4 ± 2.6 | 9.4 | 6 | 67.2 ± 3.4 | 260.5 ± 13.6 | 3.9 |
| GTM686-03 | 4 | 0.2 ± 0 | 25.2 ± 9.6 | 126 | 4 | 102.8 ± 1.4 | 258.3 ± 28.1 | 2.5 |
| GTM730-03 | 4 | 0.8 ± 0.1 | 26.2 ± 1.6 | 32.8 | 4 | 122.5 ± 10.7 | 268.8 ± 3.9 | 2.2 |
| SGTM1233-99 | 9 | 4 ± 0.9 | 12.2 ± 2.1 | 3.1 | 6 | 88.5 ± 1.8 | 256.5 ± 35.4 | 2.9 |
| SGTM2697 | 6 | 8.2 ± 2.1 | 19.5 ± 2 | 2.4 | | | | |

In view of the results summarized above, an appropriate conclusion is that IOP levels may be effectively controlled and glaucoma treated with compositions and methods comprising and using compounds with a modulating effect on ALK5 receptor activity.

Selective modulator compounds used according to certain embodiments of the present invention can be incorporated into various types of ophthalmic formulations for delivery. The compounds may be delivered directly to the eye (for example: topical ocular drops or ointments; slow release devices in the cul-de-sac or implanted adjacent to the sclera or within the eye; periocular, conjunctival, sub-tenons, intracameral, intravitreal, or intracanalicular injections). In certain embodiments, compounds may be delivered systemically (for example: orally; intravenous, subcutaneous or intramuscular injections; parenterally; dermal or nasal delivery) using techniques well known by those of ordinary skill in the art. It is fuirther contemplated that the agents of the invention may be formulated in intraocular insert or implant devices.

In preferred embodiments, selective modulator compounds according to the present invention are incorporated into topical ophthalmic formulations for delivery to the eye. The compounds may be combined with ophthalmologically acceptable preservatives, surfactants, viscosity enhancers, penetration enhancers, buffers, sodium chloride, and/or water to form an aqueous, sterile ophthalmic suspension or solution. Ophthalmic solution formulations may be prepared by dissolving a compound in a physiologically acceptable isotonic aqueous buffer. Further, the ophthalmic solution may include an ophthalmologically acceptable surfactant to assist in dissolving the compound. The ophthalmic solution may also contain an agent to increase viscosity, such as, hydroxymethylcellulose, hydroxyethylcellulose, hydroxypropylmethylcellulose, methylcellulose, polyvinylpyrrolidone, or the like, to improve the retention of the formulation in the conjunctival sac. Gelling agents can also be used, including, but not limited to, gellan and xanthan gum.

In order to prepare sterile ophthalmic ointment formulations, a selective modulator compound is combined with a preservative in an appropriate vehicle, such as, mineral oil, liquid lanolin, or white petrolatum. Sterile ophthalmic gel formulations may be prepared by suspending the compound in a hydrophilic base prepared from the combination of, for example, carbopol-974, or the like, according to the published formulations for analogous ophthalmic preparations; preservatives and tonicity agents can be incorporated.

In certain embodiments, selective modulator compounds are preferably formulated as topical ophthalmic suspensions or solutions, with a pH of about 4 to 8. The compounds will normally be contained in these formulations in an amount 0.01 to 5 percent by weight/volume (“w/v %”), but preferably in an amount of 0.25 to 2 by w/v %. A typical dosage regimen will comprise administration of 1 to 2 drops of these formulations to the surface of the eye 1 to 4 times per day, in accordance with the discretion of a skilled clinician.

The selective modulator compounds can also be used in combination with other agents for treating glaucoma, such as, but not limited to, β-blockers, prostaglandin analogs, carbonic anhydrase inhibitors, α₂ agonists, miotics, and neuroprotectants.

Certain embodiments of the present invention comprise in vitro methods of screening selective modulators of ALK5 receptor activity for the treatment of glaucoma and control of IOP. In general, these embodiments comprise culturing a plurality of TM cells in a suitable medium. TM cells may be cultured in certain embodiments according to the TM culture procedure described in the description for FIG. 3. A selective modulator of ALK5 activity is added to a first population of cultured cells. In these embodiments, a control population that does not have a selective modulator is also prepared. Then, levels of an extracellular matrix protein, such as fibronectin or PAI-1, are measured for each cell culture population in the presence and absence of TGF-β2. Any extracellular matrix proteins can be measured in embodiments of the present invention. The measured levels in a first population and in a control population are then compared. Such a comparison can be used to screen selective modulators for ALK5 receptor activity and to determine whether such selective modulators will be useful for treatment of glaucoma and control of IOP.

Shown below are several examples of pharmaceutical compositions according to embodiments of the present invention. The following examples are provided to illustrate the utility of the present invention, but should not be construed as implying any limitations to the claims.

### EXAMPLE 1

| Ingredients | Concentration (w/v %) |
|---|---|
| Compound 1 | 0.01-2% |
| Hydroxypropyl methylcellulose | 0.5% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |
| Purified water | q.s. to 100% |

### EXAMPLE 2

| Ingredients | Concentration (w/v %) |
|---|---|
| Compound 2 | 0.01-2% |
| Methyl cellulose | 4.0% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |
| Purified water | q.s. to 100% |

### EXAMPLE 3

| Ingredients | Concentration (w/v %) |
|---|---|
| Compound 13 | 0.01-2% |
| Guar gum | 0.4-6.0% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |
| Purified water | q.s. to 100% |

### EXAMPLE 4

| Ingredients | Concentration (w/v %) |
|---|---|
| Compound 12 | 0.01-2% |
| White petrolatum and mineral oil and lanolin | Ointment consistency |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein.

What is claimed is:

1. An ophthalmic pharmaceutical composition useful in the treatment of glaucoma and control of intraocular pressure comprising:
an effective amount of a selective modulator of ALK5 receptor activity.

2. The composition of claim 1 wherein said selective modulator is selected from the group consisting of: 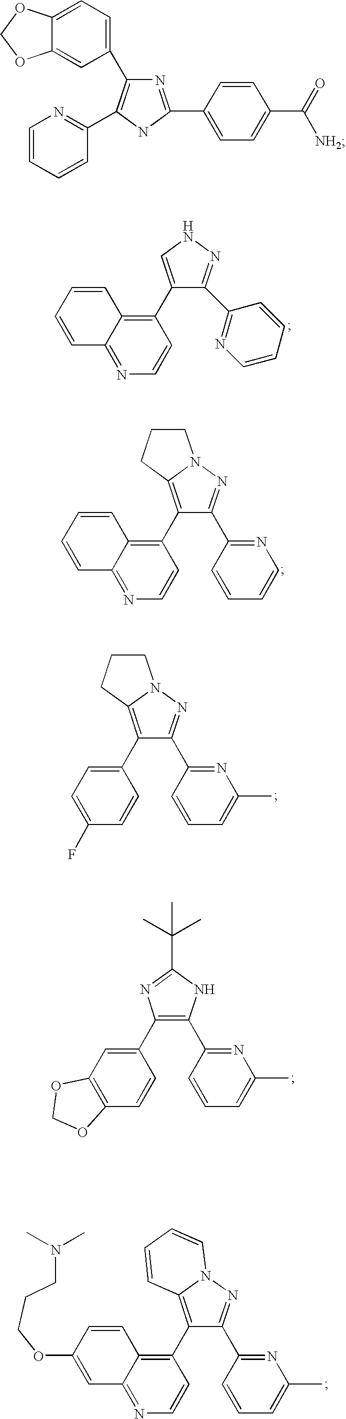 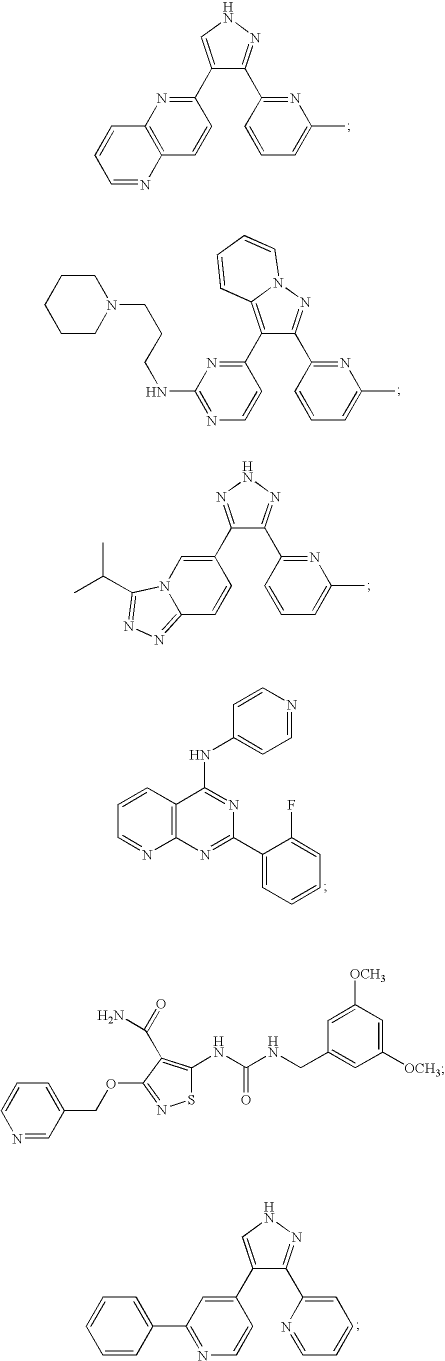 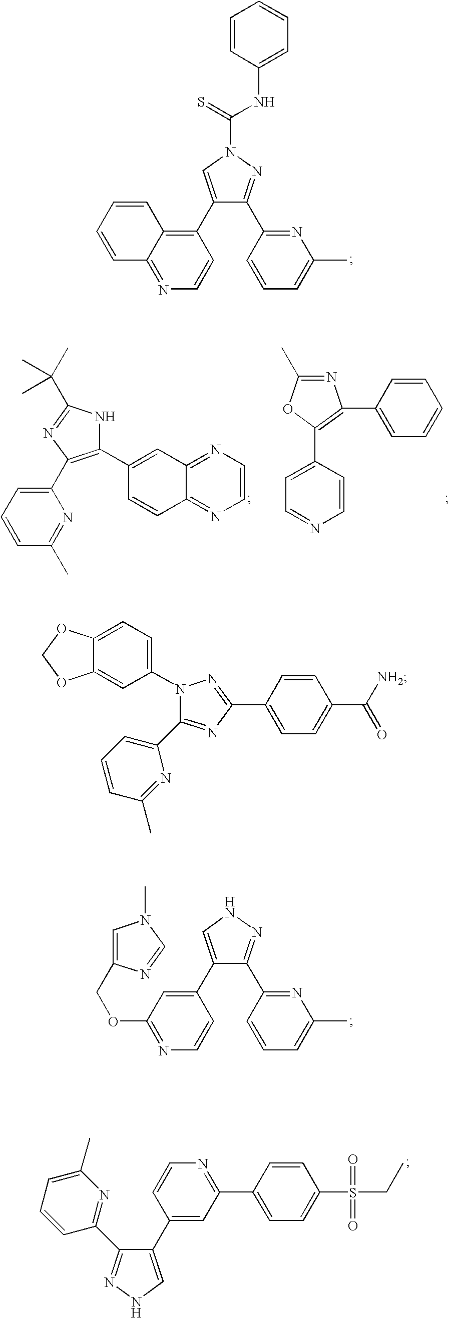 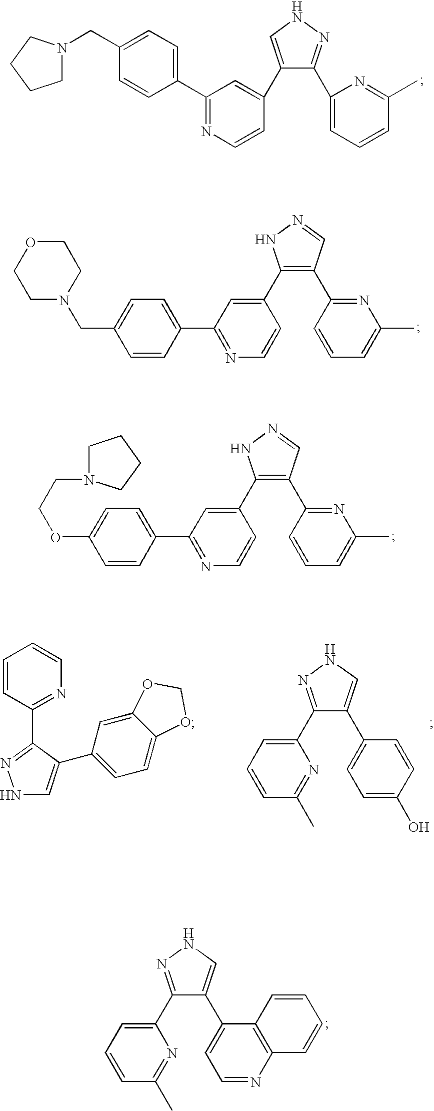
4-(3-(6-methyl pyridin-2-yl)-1H-pyrazol-4-yl)-7-ethoxy quinoline; 4-(3-pyridin-2-yl-1H-pyrazol-4-yl)-7-ethoxyquinoline; 7-fluoro-4-[3-(6-methyl-pyridin-2-yl)-1H-pyrazol-4-yl]-quinoline; 4-[3-(6-bromopyridin-2-yl)-1H-pyrazol-4-yl]-quinoline; 4-[3-(6-[n-butylamino)pyridin-2-yl]-1H-pyrazol-4-yl]-quinoline; 4-[3-(6-methylpyridin-2-yl)-1H-pyrazol-4-yl]-quinoline; 6-chloro-4-[3-(6-methylpyridin-2-yl)-1H-pyrazol-4-yl]-quinoline; 6-trifluoromethyl-4-[3-(6-methylpyridin-2-yl)-1H-pyrazol-4-yl]-quinoline; 7-methyl-4-[3-(6-methylpyridin-2-yl)-1H-pyrazol-4-yl]-quinoline; 6-methoxy-4-[3-1H-pyrazol-4-yl]-quinoline; 6-trifluoromethoxy-4-[3-(6-methylpyridin-2-yl)-1H-pyrazol-4-yl]-quinoline; 4-[3-(3-chlorophenyl)-1H-pyrazol-4-yl]-quinoline; 6-butoxy-4-(3-pyridin-2-yl-1H-pyrazol-4-yl)-quinoline; 6-sec-butyl-4-(3-pyridin-2-yl-1H-pyrazol-4-yl)-quinoline; 5-methyl-3-(6-methylpyridin-2-yl)-4-(-4-fluorophenyl)-1H-pyrazole; 4-(4-methoxyphenyl)-5-methyl-3-(6-methylpyridin-2-yl)-1H-pyrazole; 4-[5-methyl-3-(6-methylpyridin-2-yl)-1H-pyrazol-4-yl]-quinoline; 4-[3-(6-propylpyridin-2-yl)-1H-pyrazol-4-yl]-quinoline; 3-cyclopropyl-5-pyridin-2-yl-4-quinolin-4-yl-pyrazole; 3-(3-trifluoromethylphenyl)-4-quinolin-4-yl-pyrazole; 1-benzyl-3-(2-pyridyl)-4-(4-quinolyl)pyrazole; 1-(4-phenylbutyl)-3-(2-pyridyl)-4-(4-quinolyl)pyrazole; 2-(3-(2-pyridyl)-4-(4-quinolyl)pyrazolyl)ethan-1-ol; 2-(3-(2-pyridyl)-4-(4-quinolyl)pyrazolyl)ethyl methylsulfonate; 4-[2-(3-(2-pyridyl)-3-(4-quinolyl)-pyrazolyl)ethyl]morpholine; phenyl[2-(3-(2-pyridyl)-4-(4-quinolyl)-pyrazolyl)ethyl]amine; 4-(4-pyridin-2-yl-1H-pyrazol-3-yl)-quinoline; and 4-(3-pyridin-2-yl-1H-pyrazol-4-yl)-quinoline; 5-[5-(6-methylpyridin-2-yl)-1H-[1,2,3]triazol-4-yl]-benzo[1,2,5]thiadiazole; 5-[2-ethyl-5-(6-methylpyridin-2yl)-2H-[1,2,3]triazol-4-yl]-benzo[1,2,5]thiadiazole; 6-[5-(6-methylpyridin-2-yl)-1H-[1,2,3]triazol-4-yl]-[1,2,4]triazolo[1,5-a]pyridine; 2-[5-(2,3-dihydrobenzofuran-5-yl)-3H-[1,2,3]triazol-4-yl]-6-methylpyridine; 2-[5-(2,3-dihydrobenzo[1,4]dioxin-6-yl)-2H-[1,2,3]triazol-4-yl]-6-methylpyridine; 1 -methyl-6-[5-(6-methylpyridin-2-yl)-2H-[1,2,3]triazol-4-yl]-1H-benzimidazole; 6-(2-ethyl-5-(6-methylpyridin-2-yl)-2H-[1,2,3]triazol-4-yl)-[1,2,4]triazolo[1,5-a]pyridine; 6-(2-methyl-5-(6-methylpyridin-2-yl)-2H-[1,2,3]triazol-4-yl)-[1,2,4]triazolo[1,5-a]pyridine; 2-[5-(4Methoxyphenyl)-2H-[1,2,3]triazol-4-yl]-6-methylpyridine; 2-[5-(3-fluoro-4-methoxyphenyl)-2H-[1,2,3]triazol-4-yl]-6-methylpyridine; and 2-[5-(3-chloro-4-methoxyphenyl)-2H-[1,2,3]triazol-4-yl]-6-methylpyridine.

3. The composition of claim 1 comprising a pharmaceutically acceptable salt of said selective modulator.

4. The composition of claim 1 further comprising a compound selected from the group consisting of:
ophthalmologically acceptable preservatives, surfactants, viscosity enhancers, penetration enhancers, gelling agents, hydrophobic bases, vehicles, buffers, sodium chloride, and water.

5. The composition of claim 1 further comprising a glaucoma treatment agent.

6. The composition of claim 5 wherein said glaucoma treatment agent is selected from the group consisting of:
blockers, prostaglandin analogs, carbonic anhydrase inhibitors, α2 agonists, miotics, and neuroprotectants.

7. The composition of claim 1 wherein said composition comprises from about 0.01 percent weight/volume to about 5 percent weight/volume of said compound.

8. The composition of claim 1 wherein said composition comprises from about 0.25 percent weight/volume to about 2 percent weight/volume of said compound.

9. The composition of claim 1, wherein said composition further comprises a preservative, tonicity agent, antioxidant, stabilizer, wetting agent, clarifying agent or a viscosity-increasing agent.

10. An in vitro method of screening a selective modulator of ALK5 receptor activity for the treatment of glaucoma and control of intraocular pressure comprising:
culturing a plurality of trabecular meshwork (TM) cells in a suitable medium;
adding said selective modulator to a first population of said TM cells; and
comparing measured levels of an extracellular matrix-related protein in said first population and in a control population.

11. The method of claim 10 wherein said extracellular matrix-related protein is selected from the group consisting of:
fibronectin, plasminogen activator inhibitor I (PAI-1), collagens, fibrillin, vitronectin, laminin, thrombospondin I, proteoglycans, and integrins.

12. A method of treating glaucoma and controlling intraocular pressure comprising:
applying a therapeutically effective amount of a pharmaceutical composition comprising a selective modulator of ALK5 receptor activity to an affected eye of a patient.

13. The method of claim 12 wherein said applying comprises: applying a composition of claim 2.

14. The method of claim 13 wherein said applying comprises applying using a technique selected from the group consisting of:
periocular injection, conjunctival injection, sub-tenons injection, intracameral injection, intravitreal injection, intracanalicular injection, implanting delivery device in the cul-de-sac, implanting delivery device adjacent to the sclera, implanting delivery device within the eye, oral administration, intravenous administration, subcutaneous administration, intramuscular administration, parenteral administration, dermal administration, and nasal administration.

15. The method of claim 12, wherein said pharmaceutical composition comprises a preservative, tonicity agent, antioxidant, stabilizer, wetting agent, clarifying agent or a viscosity-increasing agent.
